(19) United States
(12) Patent Application Publication
(10) Pub. No.: US 2024/0147990 A1
(43) Pub. Date: May 9, 2024

(54) Delivery Particles Based On Amine-Thiol-Ene Conjugates and Derivatives

(71) Applicant: Encapsys, LLC, Appleton, WI (US)

(72) Inventors: TODD ARLIN SCHWANTES, Appleton, WI (US); Terri Anne Marquardt, Appleton, WI (US)

(73) Assignee: Encapsys, LLC, Appleton, WI (US)

(21) Appl. No.: 18/475,472

(22) Filed: Sep. 27, 2023

Related U.S. Application Data
(60) Provisional application No. 63/418,196, filed on Oct. 21, 2022.

(51) Int. Cl. A01N 25/28; A01P 7/04; A01P 13/00; A61K 8/11

(52) U.S. Cl. CPC A01N 25/28; A01P 7/04; A01P 13/00; A61K 8/11

(57) ABSTRACT

An improved delivery particle comprising a benefit agent core material and a shell encapsulating the core material is described, along with a process for forming such a delivery particle and articles of manufacture. The shell is the reaction product of an ATEC (amine-thiol ene) conjugate, which can be further reacted with one or more multifunctional (meth)acrylates or isocyanates, or both. The delivery particle of the invention has improved release characteristics, and embodiments exhibit enhanced degradation characteristics in OECD test method 301B

### FIELD OF THE INVENTION

This invention relates to capsule manufacturing processes and biodegradable delivery particles produced by such processes, the delivery particles containing a core material and a shell encapsulating the core.

### DESCRIPTION OF THE RELATED ART

Microencapsulation is a process where droplets of liquids, particles of solids or gasses are enclosed inside a solid shell and are generally in the micro-size range. The core material is separated from the surrounding environment by the shell. Microencapsulation technology has a wide range of commercial applications for different industries. Overall, capsules are capable of one or more of (i) providing stability of a formulation or material via the mechanical separation of incompatible components, (ii) protecting the core material from the surrounding environment, (iii) masking or hiding an undesirable attribute of an active ingredient and (iv) controlling or triggering the release of the active ingredient to a specific time or location. All of these attributes can lead to an increase of the shelf-life of several products and a stabilization of the active ingredient in liquid formulations.

Various processes for microencapsulation, and exemplary methods and materials are set forth in Schwantes (U.S. Pat. No. 6,592,990), Nagai et al. (U.S. Pat. No. 4,708,924), Baker et al. (U.S. Pat. No. 4,166,152), Wojciak (U.S. Pat. No. 4,093,556), Matsukawa et al. (U.S. Pat. No. 3,965,033), Ozono (U.S. Pat. No. 4,588,639), Irgarashi et al. (U.S. Pat. No. 4,610,927), Brown et al. (U.S. Pat. No. 4,552,811), Scher (U.S. Pat. No. 4,285,720), Jahns et al. (U.S. Pat. Nos. 5,596,051 and 5,292,835), Matson (U.S. Pat. No. 3,516,941), Foris et al. (U.S. Pat. Nos. 4,001,140; 4,087,376; 4,089,802 and 4,100,103), Greene et al. (U.S. Pat. Nos. 2,800,458; 2,800,457 and 2,730,456), Clark (U.S. Pat. No. 6,531,156), Hoshi et al. (U.S. Pat. No. 4,221,710), Hayford (U.S. Pat. No. 4,444,699), Hasler et al. (U.S. Pat. No. 5,105,823), Stevens (U.S. Pat. No. 4,197,346), Riecke (U.S. Pat. No. 4,622,267), Greiner et al. (U.S. Pat. No. 4,547,429), and Tice et al. (U.S. Pat. No. 5,407,609), among others and as taught by Herbig in the chapter entitled “Microencapsulation” in Kirk-Othmer Encyclopedia of Chemical Technology, V.16, pages 438-463.

Core-shell encapsulation is useful to preserve actives, such as benefit agents, in harsh environments and to release them at the desired time, which may be during or after use of goods incorporating the encapsulates. Among various mechanisms that can be used for release of benefit agent from the encapsulates, the one commonly relied upon is mechanical rupture of the capsule shell through friction or pressure. Selection of mechanical rupture as the release mechanism constitutes another challenge to the manufacturer, as rupture must occur at specific desired times, even if the capsules are subject to mechanical stress prior to the desired release time.

Industrial interest for encapsulation technology has led to the development of several polymeric capsules chemistries which attempt to meet the requirements of biodegradability, low shell permeability, high deposition, targeted mechanical properties, and rupture profile. Increased environmental concerns have put the polymeric capsules under scrutiny, therefore manufacturers have started investigating sustainable solutions for the encapsulation of benefit agents.

Biodegradable materials exist and are able to form delivery particles via coacervation, spray-drying or phase inversion precipitation. However, the delivery particles formed using these materials and techniques are highly porous and not suitable for aqueous compositions containing surfactants or other carrier materials, since the benefit agent is prematurely released to the composition.

Non-leaky and performing delivery particles in aqueous surfactant-based compositions exist, however due to its chemical nature and cross-linking, they are not biodegradable.

Encapsulation can be found in areas as diverse as pharmaceuticals, personal care, textiles, food, coatings, and agriculture. In addition, the main challenge faced in encapsulation is that a complete retention of the encapsulated active within the capsule is required throughout the whole supply chain, until a controlled or triggered release of the core material is applied. There are significantly limited microencapsulation technologies that can fulfill the rigorous criteria for long-term retention and active protection capability for commercial needs, especially when it comes to encapsulation of small molecules.

Delivery particles are needed that have high structural integrity so as to reduce leakage and resist damage from harsh environments. Variations of such delivery particles able to be designed to be biodegradable as taught herein would solve the problem of sustainability.

### Definitions

As used herein, reference to the term “(meth)acrylate” or “(meth)acrylic” is to be understood as referring to both the acrylate and the methacrylate versions of the specified monomer, oligomer and/or prepolymer, (for example “isobornyl (meth)acrylate” indicates that both isobornyl methacrylate and isobornyl acrylate are possible, similarly reference to alkyl esters of (meth)acrylic acid indicates that both alkyl esters of acrylic acid and alkyl esters of methacrylic acid are possible, similarly poly(meth)acrylate indicates that both polyacrylate and polymethacrylate are possible). Similarly, the use of the phrase “prepolymer” means that the referenced material may exist as a prepolymer or combination of oligomers and prepolymers. Similarly, it is to be understood that the general reference herein to (meth)acrylate or (meth)acrylates, e.g., “water soluble (meth)acrylates,” “water phase (meth)acrylate,” etc., is intended to cover or include the (meth)acrylate monomers and/or oligomers. Additionally, the descriptors “water soluble or dispersible,” “water soluble,” and “water dispersible” when referencing certain (meth)acrylate monomers and/or oligomers or initiators means that the specified component is soluble or dispersible in the given matrix solution on its own or in the presence of a suitable solubilizer or emulsifier or upon attainment of certain temperatures and/or pH.

Each alkyl moiety herein, unless otherwise indicated, can be from C₁ to C₈, or even from C₁ to C₂₄. Poly(meth)acrylate materials are intended to encompass a broad spectrum of polymeric materials including, for example, polyester poly(meth)acrylates, urethane and polyurethane poly(meth)acrylates (especially those prepared by the reaction of a hydroxyalkyl (meth)acrylate with a polyisocyanate or a urethane polyisocyanate), methyl cyanoacrylate, ethyl cyanoacrylate, diethylene glycol di(meth)acrylate, trimethylolpropane tri(meth)acrylate, ethylene glycol di(meth)acrylate, allyl (meth)acrylate, glycidyl (meth)acrylate, (meth)acrylate functional silicones, di-, tri- and tetraethylene glycol di(meth)acrylate, dipropylene glycol di(meth)acrylate, polyethylene glycol di(meth)acrylate, di(pentamethylene glycol) di(meth)acrylate, ethylene di(meth)acrylate, neopentyl glycol di(meth)acrylate, ethoxylated bisphenol A di(meth)acrylates, bisphenol A di(meth)acrylates, diglycerol di(meth)acrylate, tetraethylene glycol dichloroacrylate, 1,3-butanediol di(meth)acrylate, neopentyl di(meth)acrylate, polyethylene glycol di(meth)acrylate and dipropylene glycol di(meth)acrylate and various multifunctional (meth)acrylates and multifunctional amine (meth)acrylates. Monofunctional acrylates, i.e., those containing only one acrylate group, may also be advantageously used. Typical monoacrylates include 2-ethylhexyl (meth)acrylate, 2-hydroxyethyl (meth)acrylate, cyanoethyl (meth)acrylate, 2-hydroxypropyl (meth)acrylate, p-dimethyl aminoethyl (meth)acrylate, lauryl (meth)acrylate, cyclohexyl (meth)acrylate, tetrahydrofurfuryl (meth)acrylate, chlorobenzyl (meth)acrylate, amino alkyl(meth)acrylate, various alkyl(meth)acrylates and glycidyl (meth)acrylate. Of course, mixtures of (meth)acrylates or their derivatives as well as combinations of one or more (meth)acrylate monomers, oligomers and/or prepolymers or their derivatives with other copolymerizable monomers, including acrylonitriles and methacrylonitriles may be used as well. Multifunctional (meth)acrylate monomers will typically have at least two, at least three, and preferably at least four, at least five, or even at least six polymerizable functional groups.

For ease of reference in this specification and in the claims, the term “monomer” or “monomers” as used herein with regard to the structural materials that form the wall polymer of the delivery particles is to be understood as monomers, but also is inclusive of oligomers and/or prepolymers formed of the specific monomers.

As used herein the term “water soluble material” means a material that has a solubility of at least 0.5% wt in water at 60° C.

As used herein the term “oil soluble” means a material that has a solubility of at least 0.1% wt in the core of interest at 50° C.

As used herein the term “oil dispersible” means a material that can be dispersed at least 0.1% wt in the core of interest at 50° C. without visible agglomerates.

As used herein, the terms “delivery particle” and “microcapsule” or “encapsulate” are used interchangeably. “Wall material” or “shell” are also used interchangeably to refer to the shell of the delivery particle. “Benefit agent” or “core” are used interchangeably to refer to the contents encapsulated within the delivery particle.

### SUMMARY OF THE INVENTION

The invention describes a delivery particle comprising a core material and a shell encapsulating the core material. The core material can comprise a benefit agent. The shell comprises a polymer. More particularly, the polymer comprises the reaction product of:
an amine-thiol-ene conjugate. In alternative embodiments, the amine-thiol-ene conjugate in addition is crosslinked with one or more of
a multifunctional (meth)acrylate; or,
an isocyanate.

Articles of manufacture based on such delivery particles are also described.

### DETAILED DESCRIPTION

The invention describes a novel delivery particle. The delivery particle comprises a core material and a shell encapsulating the core material. The core material comprises a benefit agent. The shell comprises a polymer. More particularly, the polymer comprises the reaction product of:
an amine-thiol-ene conjugate. The conjugate in addition can be further crosslinked with one or more of a multifunctional (meth)acrylate or an isocyanate, as more fully described herein.

In embodiments, the amine of the amine-thiol-ene conjugate (“ATEC”) forming the delivery particle shell is a mono or bis alkyl diamine, or mono or bis alkyl triamine. Amines can be primary amines and by way of illustration and not limitation, can include furfurylamine, allylamine, dimethylene diamine, n-butylamine, octylamine, propylamine, ethylamine, benzylamine, As more fully described herein and in the examples, useful amines in the amine-thiol-ene conjugation are primary amines and aliphatic amines with electron withdrawing groups. Secondary amines can be useful for further reaction of the amine-thiol-ene conjugate with acrylates. Multifunctional amines can be employed to introduce additional side chain reactive groups. Electron withdrawing groups can include nitro (—NO₂), aldehyde (—CHO), ketone (—C═OR), cyano (—CN), carboxylic (—COOH) or ester (—COOR).

In more specific embodiments the thiol of the amine-thiol-ene conjugate is a thiolactone. The thiolactone ring can typically have from 3 to 8 members, consisting of a thiol group and carbons. The thiolactone ring can be substituted with amine groups, such as primary amines. The thiolactone can comprise homocysteine thiolactone or acetylated homocysteine thiolactone.

The ene portion of the amine-thiol-ene conjugate is an unsaturated compound and can be selected from a vinyl compound or an acrylate. In embodiments, where a vinyl compound is selected as the ene portion of the amine-thiol-ene conjugate, the vinyl compound beneficially includes a functional group comprising an electron withdrawing group. The ene is an unsaturated compound having an olefinic bond, and addition of the ene to the conjugate proceeds via the olefinic bond. The molar ratio of the amine to thiol to ene moieties in the conjugate is from 0.75/1/1.25 to 1.25/1/0.75 preferably 0.85/1/1.15 to 1.15/1/0.85 more preferably 0.95/1/1.05 to 1.05/1/0.95.

The amine-thiol-ene conjugate by further combination can comprise in addition a free-radically crosslinked reaction product of the amine-thiol-ene conjugate with one or more of a multifunctional (meth)acrylate and an isocyanate. For clarity, the amine-thiol-ene conjugate can be further reacted with the multifunctional (meth)acrylate or can be reacted with isocyanate or can be reacted with both, making for versatility in shell polymer design of the delivery particle.

The amine-thiol-ene conjugate reacted with an isocyanate can be further free-radically crosslinked with a multifunctional (meth)acrylate. In variations, the amine-thiol-ene conjugate can be further reacted and in addition can form a reaction product of the amine-thiol-ene conjugate with either of an isocyanate or a multifunctional (meth)acrylate, or even both. This permits considerable versatility in polymer assembly for specific purposes. In embodiments, the multifunctional (meth)acrylate is present in a molar excess as compared to the thiolactone and amine.

The amine-thiol-ene conjugate can comprise a further crosslinked reaction product of the amine-thiol-ene conjugate with one or more of an isocyanate, or a Michael adduct comprising a multifunctional (meth)acrylate, or one or more of an aza-Michael adduct comprising a multifunctional acrylate. In various alternatives, the amine-thiol-ene conjugate can comprise the minor or major constituent of the polymer, desirably at least 50% by weight of the shell composition. Advantageously the amine thiol ene conjugate can be the either the sole polymer (100%) or can be combined as a blend with a portion of the one or more of isocyanate or (meth)acrylate. In alternative embodiments the isocyanate and/or methacrylate forms from 0 to 10%, or even from 0 to 25% or even from 0 to 50% or even from 15 to 50% of the polymer.

The delivery particle comprises a core material and a shell encapsulating the core material, wherein the core material comprises a benefit agent, and, wherein the shell comprises a polymer, the polymer comprising a reaction product of an amine, a thiol lactone, a multifunctional (meth)acrylate and a polyfunctional. In embodiments it should be understood that additional combinations can be assembled by further reacting the amine thio ene conjugate by addition of isocyanate alone, (meth)acrylate alone, or a combination of the two.

In embodiments, the amine-thiol-ene conjugate is the reaction product of an alkyl amine, a 3-, 4-, 5-, or 6-member thiolactone, preferably a 4-, 5-, or 6-member thiolactone, and a vinyl compound having an electron withdrawing group. The delivery particle composition, where in the ATEC conjugate the amine is alkyl amine, the alkyl moiety can be selected from a 3 to 18 carbon alkyl moiety. Electron withdrawing substituents on the vinyl compound can include any of nitro (NO₂), aldehyde (—CHO), ketone (—C═OR), cyano (—CN), carboxylic (—COOH) or ester (—COOR).

In delivery particle compositions, (meth)acrylate can be multifunctional (meth)acrylate and can be selected from an oil soluble (meth)acrylate selected from group consisting of a bi-functional (meth)acrylate, a tri-functional (meth)acrylate, a tetra-functional (meth)acrylate, a penta-functional (meth)acrylate, a hexa-functional (meth)acrylate, a hepta-functional (meth)acrylate, an octa-functional (meth)acrylate and mixtures thereof.

Optionally, the multifunctional (meth)acrylate is selected from a water soluble or dispersible (meth)acrylate selected from 2-carboxyethyl acrylate, 2-carboxyethyl acrylate oligomers, 2-carboxypropyl acrylate, 4-acryloyloxyphenylacetic acid, carboxyoctyl acrylate, tripropylene glycol diacrylate, ethoxylated bisphenol diacrylate, dipropylene glycol diacrylate, alkoxylated hexanediol diacrylate, alkoxylated cyclohexane dimethanol diacrylate, propoxylated neopentyl glycol diacrylate, trimethylolpropane triacrylate, pentaerythritol triacrylate, ethoxylated trimethylolpropane triacrylate, propoxylated trimethylolpropane triacrylate, propoxylated glyceryl triacrylate, ditrimethylolpropane tetraacrylate, dipentaerythritol pentaacrylate, ethoxylated pentaerythritol tetraacrylate, glycerol tri(meth)acrylate, ethylene glycol diacrylate, di-, tri-, tetra-, or pentaethylene glycol diacrylate, dipropylene glycol diacrylate, polyethylene glycol diacrylate, 2-ethylhexyl acrylate, 2-hydroxyethyl (meth)acrylate, cyanoethyl acrylate, 2-hydroxypropyl acrylate, lauryl acrylate, cyclohexyl acrylate, tetrahydrofurfuryl acrylate, chlorobenzyl acrylate, amino alkylacrylate, ethylaminoethyl (meth)acrylate, aminoethyl (meth)acrylate, tertiarybutyl aminoethyl (meth)acrylate, diethylamino (meth)acrylate, diethylaminoethyl (meth)acrylate, dimethylaminoethyl (meth)acrylate independently or a combination of the foregoing.

The core constitutes the material encapsulated by the microcapsules. Typically, particularly when the core material is a liquid material, the core material is combined with one or more of the compositions from which the internal wall of the microcapsule is formed or solvent for the benefit agent or partitioning modifier. If the core material can function as the oil solvent in the capsules, e.g., acts as the solvent or carrier for either the wall forming materials or benefit agent, it is possible to make the core material the major material encapsulated, or if the carrier itself is the benefit agent, can be the total material encapsulated. Usually however, the benefit agent is from 0.01 to 99 weight percent of the capsule internal contents, preferably 0.01 to about 65 by weight of the capsule internal contents, and more preferably from 0.1 to about 45% by weight of the capsule internal contents. With certain applications, the core material can be effective even at just trace quantities.

Where the benefit agent is not itself sufficient to serve as the oil phase or solvent, particularly for the wall forming materials, the oil phase can comprise a suitable carrier and/or solvent. In this sense, the oil is optional, as the benefit agent itself can at times be the oil. These carriers or solvents are generally an oil, preferably have a boiling point greater than about 80° C. and low volatility and are non-flammable. Though not limited thereto, they preferably comprise one or more esters, preferably with chain lengths of up to 18 carbon atoms or even up to 42 carbon atoms and/or triglycerides such as the esters of C6 to C12 fatty acids and glycerol. Exemplary carriers and solvents include, but are not limited to: ethyldiphenylmethane; isopropyl diphenylethane; butyl biphenyl ethane; benzylxylene; alkyl biphenyls such as propylbiphenyl and butylbiphenyl; dialkyl phthalates e.g. dibutyl phthalate, dioctylphthalate, dinonyl phthalate and ditridecylphthalate; 2,2,4-trimethyl-1,3-pentanediol diisobutyrate; alkyl benzenes such as dodecyl benzene; alkyl or aralkyl benzoates such as benzyl benzoate; diaryl ethers; di(aralkyl)ethers and aryl aralkyl ethers; ethers such as diphenyl ether, dibenzyl ether and phenyl benzyl ether; liquid higher alkyl ketones (having at least 9 carbon atoms); alkyl or aralkyl benzoates, e.g., benzyl benzoate; alkylated naphthalenes such as dipropylnaphthalene; partially hydrogenated terphenyls; high-boiling straight or branched chain hydrocarbons; alkaryl hydrocarbons such as toluene; vegetable and other crop oils such as canola oil, soybean oil, corn oil, sunflower oil, cottonseed oil, lemon oil, olive oil and pine oil; methyl esters of fatty acids derived from transesterification of vegetable and other crop oils, methyl ester of oleic acid, esters of vegetable oil, e.g. soybean methyl ester, straight chain paraffinic aliphatic hydrocarbons, and mixtures of the foregoing. In certain embodiments, the benefit agent comprising the core is a fragrance, not by way of limitation, but preferably a fragrance comprising perfume raw materials characterized by a logP of from about 2.5 to about 4.5. The core can comprise in addition a partitioning modifier selected from the group consisting of isopropyl myristate, vegetable oil, modified vegetable oil, mono-, di-, and tri-esters of C4-C24 fatty acids, dodecanophenone, lauryl laurate, methyl behenate, methyl laurate, methyl palmitate, methyl stearate, and mixtures thereof, preferably isopropyl myristate. Although illustrated here with fragrance, other benefit agents, as more fully described in this specification such as agricultural actives, can comprise the core with the polymer shell of this invention.

In certain embodiments, the wall has a biodegradability above 30% CO2 in 60 days following an OECD 301B test, preferably above 40% CO2, more preferably above 50% CO2, even more preferably above 60% CO2.

Optionally or alternatively, the wall of the delivery particles further comprises a coating material, preferably wherein the coating material is selected from the group consisting of poly(meth)acrylate, poly(ethylene-maleic anhydride), polyamine, wax, polyvinylpyrrolidone, polyvinylpyrrolidone co-polymers, polyvinylpyrrolidone-ethyl acrylate, polyvinylpyrrolidone-vinyl acrylate, polyvinylpyrrolidone methacrylate, polyvinylpyrrolidone/vinyl acetate, polyvinyl acetal, polyvinyl butyral, polysiloxane, poly(propylene maleic anhydride), maleic anhydride derivatives, co-polymers of maleic anhydride derivatives, polyvinyl alcohol, styrene-butadiene latex, gelatine, gum arabic, carboxymethyl cellulose, carboxymethyl hydroxyethyl cellulose, hydroxyethyl cellulose, other modified celluloses, sodium alginate, chitosan, chitin, casein, pectin, modified starch, polyvinyl acetal, polyvinyl butyral, polyvinyl methyl ether/maleic anhydride, polyvinyl pyrrolidone and its co polymers, poly(vinyl pyrrolidone/methacrylamidopropyl trimethyl ammonium chloride), polyvinylpyrrolidone/vinyl acetate, polyvinyl pyrrolidone/dimethylaminoethyl methacrylate, polyvinyl amines, polyvinyl formamides, polyallyl amines, copolymers of polyvinyl amines, and mixtures thereof.

The invention also describes a process of forming a population of delivery particles, the delivery particles comprising a core material and a shell encapsulating the core material, wherein the core material comprises a benefit agent; and, wherein the shell comprises a polymer, the polymer comprising the reaction product of:

the process comprising:

The delivery particle has a leakage of below about 50%, as determined by the Leakage Test described in the TEST METHODS Section.

In further constructs, the delivery particles of the invention can be fashioned into new articles by incorporation into various articles of manufacture. Such article can be selected from the group consisting of an agricultural formulation, a slurry encapsulating an agricultural active, a population of dry microcapsules encapsulating an agricultural active, an agricultural formulation encapsulating an insecticide, and an agricultural formulation for delivering a pre-emergent herbicide. The agricultural active can be selected from the group consisting of an agricultural herbicide, an agricultural pheromone, an agricultural pesticide, an agricultural nutrient, an insect control agent, and a plant stimulant.

### DETAILED DESCRIPTION

The invention describes a delivery particle comprising a core material and a shell encapsulating the core material. The core material can comprise a benefit agent. The shell comprises a polymer. More particularly, the polymer comprises the reaction product of:
an amine-thiol-ene conjugate (ATEC), and in addition the ATEC conjugate is crosslinked with one or more of
i) a multifunctional (meth)acrylate; or,
ii) an isocyanate.

More particularly the delivery particle shell comprises a polymer. The polymer comprises an amine-thiol-ene conjugate which is a reaction product of an amine, a thiol lactone, and an ene compound, and in embodiments the thiol-ene-conjugate is further reacted with one or more of a multifunctional methacrylate and a polyfunctional isocyanate, or both. In the invention, combinations of more than one multifunctional methacrylate or more than one polyfunctional isocyanate can be adopted to tailor the polymer of the delivery particle shell.

Capsules were successfully prepared using ATEC conjugate alone as the polymer. However by further crosslinking with isocyanate or with acrylate monomers and prepolymers an encapsulate can be fashioned with customized characteristics advantageously benefitting from attributes of the constituent monomers or prepolymers selected.

The amine-thiol-ene (ATEC) reaction can be accomplished with a primary amine group reacting with a thiolactone compound, opening the thiolactone ring and creating an amide bond and a reactive thiol group. The formed reactive thiol group further undergoes a Michael addition with a vinyl group such as with an acrylate group, forming a repeating unit (amide/thiol/ester) of a polymer. Thiolactones are four to eight member rings, most commonly four, five and six member rings such as represented by β, γ and δ thiolactones. The γ and γ, 5- and 6-member ring thiolactones respectively, are useful in polymer synthesis. Lysis of the thiol ring by reaction with a nucleophile results in ring opening via nucleophilic addition-elimination. Nucleophiles for ring opening can include in basic conditions, water, alcohols, and amines, with amines more favored as aminolysis does not require additives. γ thiolactones can include homocysteine-γ-thiolactone or α-amino-γ-butyrothiolactone. Aminolysis of γ thiolactones can be used to produce derivatives such as N-acetylhomocysteine thiolactone or a-isocyanato-γ thiolactone, or N-(2-alkylacetyl)homocysteine thiolactone. The isocyanato lactone can be converted to carbamates, ureas and semi-carbazides in the presence of alcohols, amines or hydrazines.

Thiolactone ring opening is achieved with reaction with the nucleophile. Primary amines are especially nucleophilic and useful for this purpose. Enolates formed by the abstraction of the a-hydrogen by a strong base are also nucleophiles. Useful nucleophiles can include amines and also enolates. The amine thiol ene conjugate reaction is attractive because of the very mild conditions generally required for it to occur. Formation of enolates can involve more harsh conditions which could also potentially drive competing reactions, e.g., Michael addition to the ene compound.

The thiol groups that become available from lysis of the thiolactone are reactive with unsaturated groups, for example vinyl or acrylate groups . Reaction of the thiol groups with the unsaturated group can proceed through radical mediated thiol-acrylate reaction or thiol acrylate Michael addition.

Lysis of the thiolactone under basic conditions in the presence of an alkylating agent can yield alkylated thiolactone. Alkylation of the thiol group can give rise to alkylated homocysteine derivatives. Through sequential hydrolysis and conjugate addition, for example γ-thiobutyrolactone with propiolic acid under basic conditions generates a corresponding acrylic acid. Aminolysis is preferred as compared to alcoholysis and hydrolysis since no additives are needed. Aminolysis allows the introduction of a functional group in the reaction product via the amine.

Thiolactone aminolysis with addition of the generated thiol to an unsaturated group such as vinyl results in an amine-thiol-ene conjugate. The generated thiol can be added to an unsaturated group either by a radical process or by nucleophilic addition.

Useful amines in the amine-thiol-ene conjugation are primary amines and aliphatic amines with electron withdrawing groups. Secondary amines can be useful for further reaction of the amine-thiol-ene conjugate with acrylates. Multifunctional amines can be employed to introduce additional side chain reactive groups. Electron withdrawing groups can include nitro (—NO₂), aldehyde (—CHO), ketone (—C═OR), cyano (—CN), carboxylic (—COOH) or ester (—COOR).

If multifunctional primary amines and multifunctional acrylates are used as reactants in the ATEC conjugate, high molecular weight polymers can be prepared. Additionally, if a molar excess of acrylate functionality is used in the conjugation, the ATEC polymer can include residual acrylates groups that can be further polymerized. Following formation of the amine-thiol-ene conjugate (“ATEC conjugate”), further reaction of the ATEC conjugate with either of both of isocyanate and acrylate moieties can yield a tailored polymer.

Acrylate moieties, as the unsaturated ene in forming the conjugate, can undergo aza-Michael addition with a primary amine. Further reaction of the ATEC conjugate with remaining or additional unsaturated groups, such as acrylate or methacrylate can proceed through radical mediated reaction or Michael addition.

Reaction can proceed following ring opening via the thiol group, or in the presence of a strong base for the rest of the adduct, a Michael donor can be deprotonated forming an anion which can react with an acrylate double bond and can be used to regenerate the base. The adduct formed may even undergo a second albeit slower Michael addition. Michael donors include various amines, enolates, and thiols and even phosphines. Michael acceptors would include methacrylates, (meth)acrylamides, maleimides, acrylonitriles, and cyanoacrylates.

The following sequence schematically demonstrates assembly of an ATEC conjugate which can be employed in the encapsulation of the invention, and these are further described in the examples.

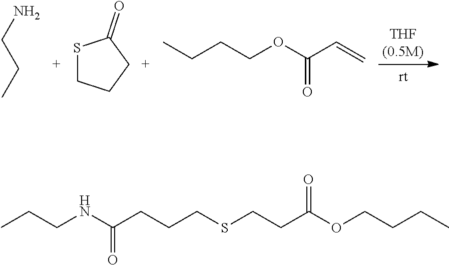

or, alternatively, amine can be diamine and thiolactone can be homocysteine thiolactone and the acrylate can be polyethyleneglycol diacrylate as shown below:

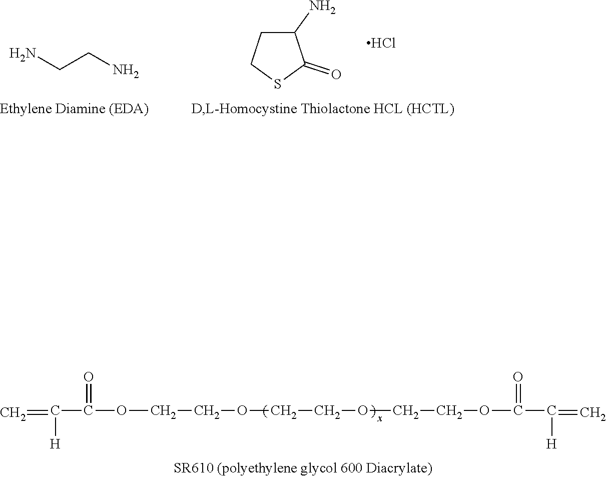

The ATEC conjugate can be further reacted with isocyanate or (meth)acrylates. For example, any remaining or additional unsaturated groups, such as acrylate or methacrylate can proceed through radical mediated reaction or Michael addition to the formed ATEC conjugate. Amines on the ATEC conjugate can be used as reaction sites with (meth)acrylate. Acrylates can also undergo Michael addition with any remaining amine groups of the ATEC conjugate. Isocyanates will also bond with any remaining primary amine groups. Generally primary amines are more reactive than secondary amines. Reaction with carboxylic acids can be used to form corresponding amides.

In forming the ATEC conjugate, the following thiolactones are illustrative:

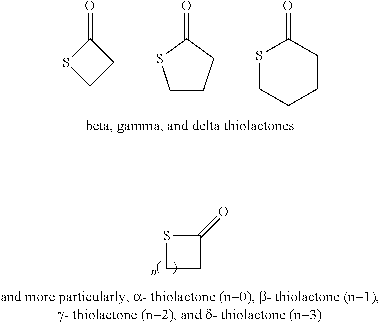

More specific examples of thiolactones that can be used to form the ATEC conjugate are:

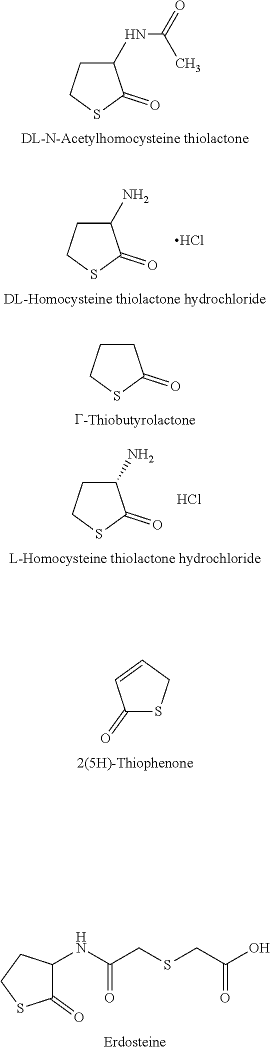

In forming the ATEC conjugate, the following amines are illustrative:

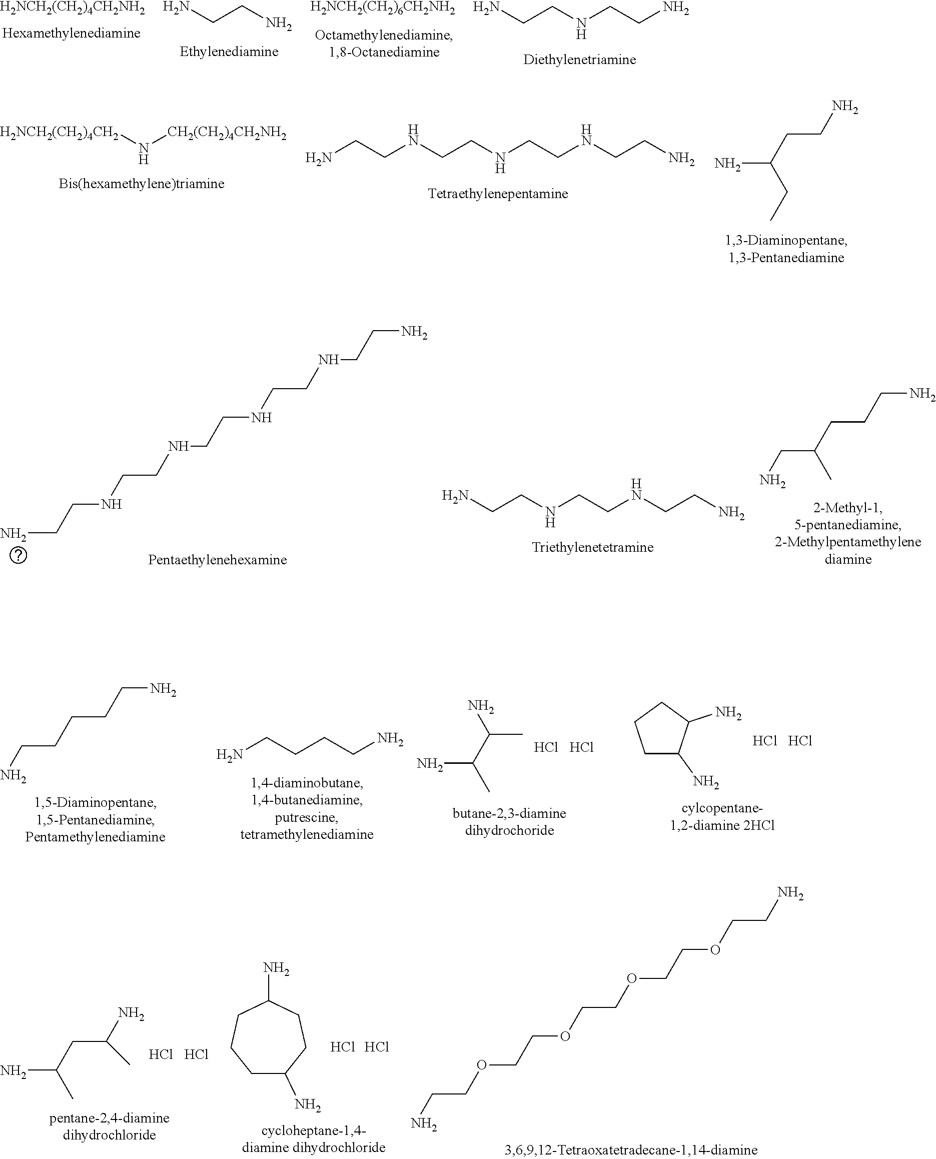

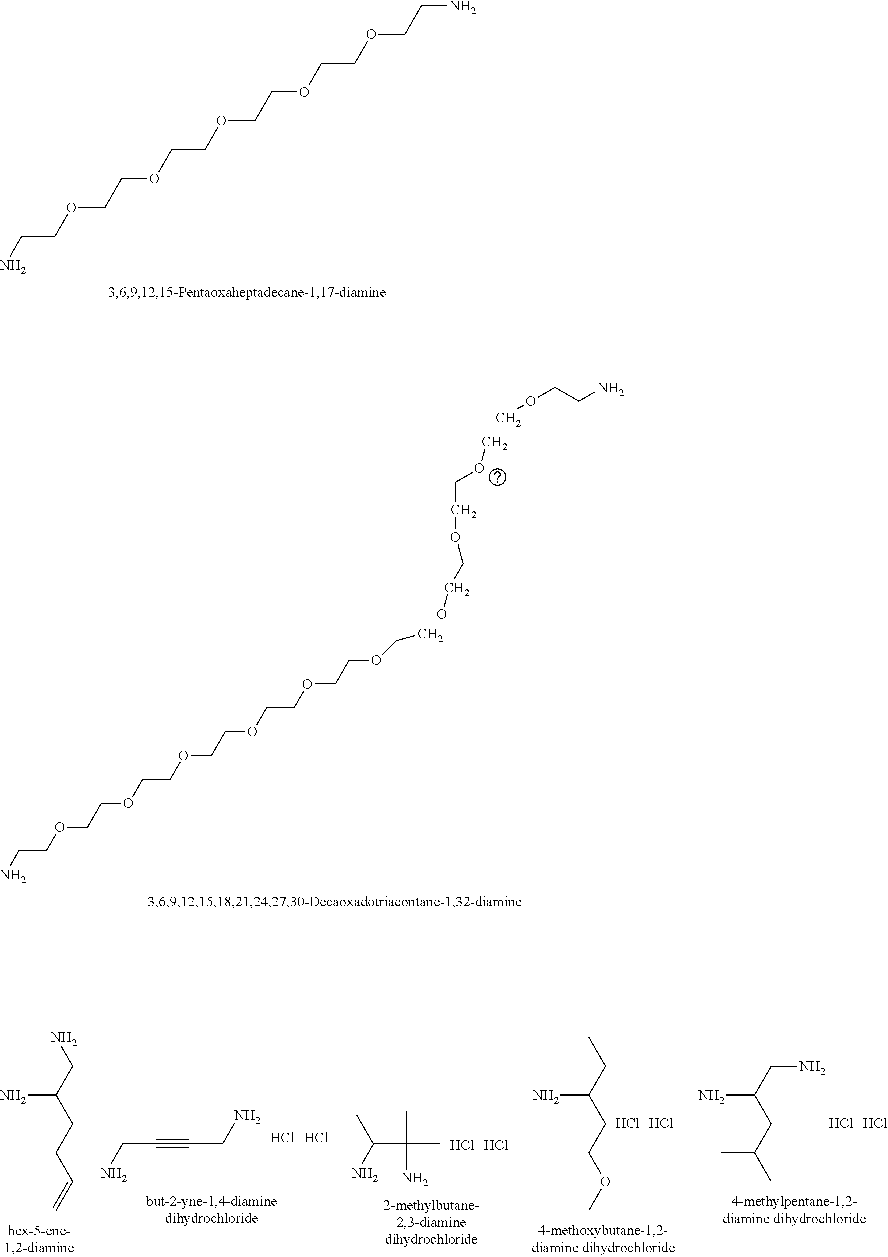

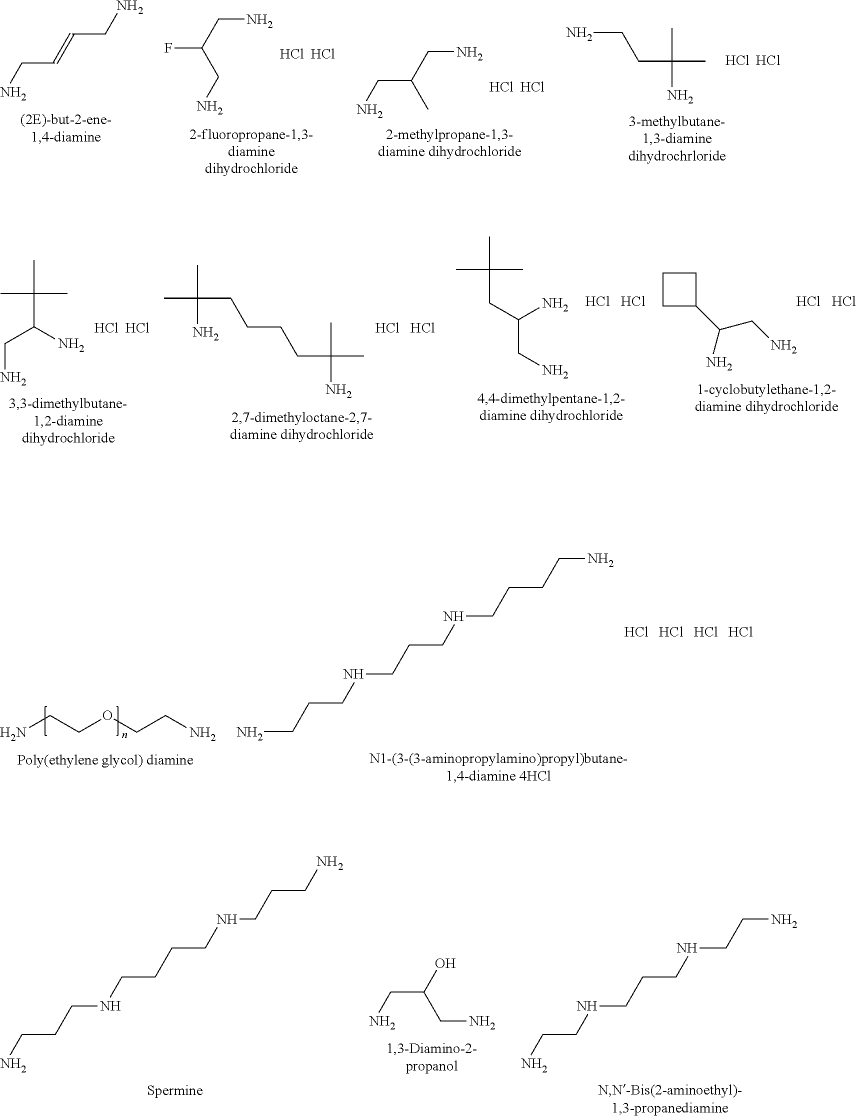

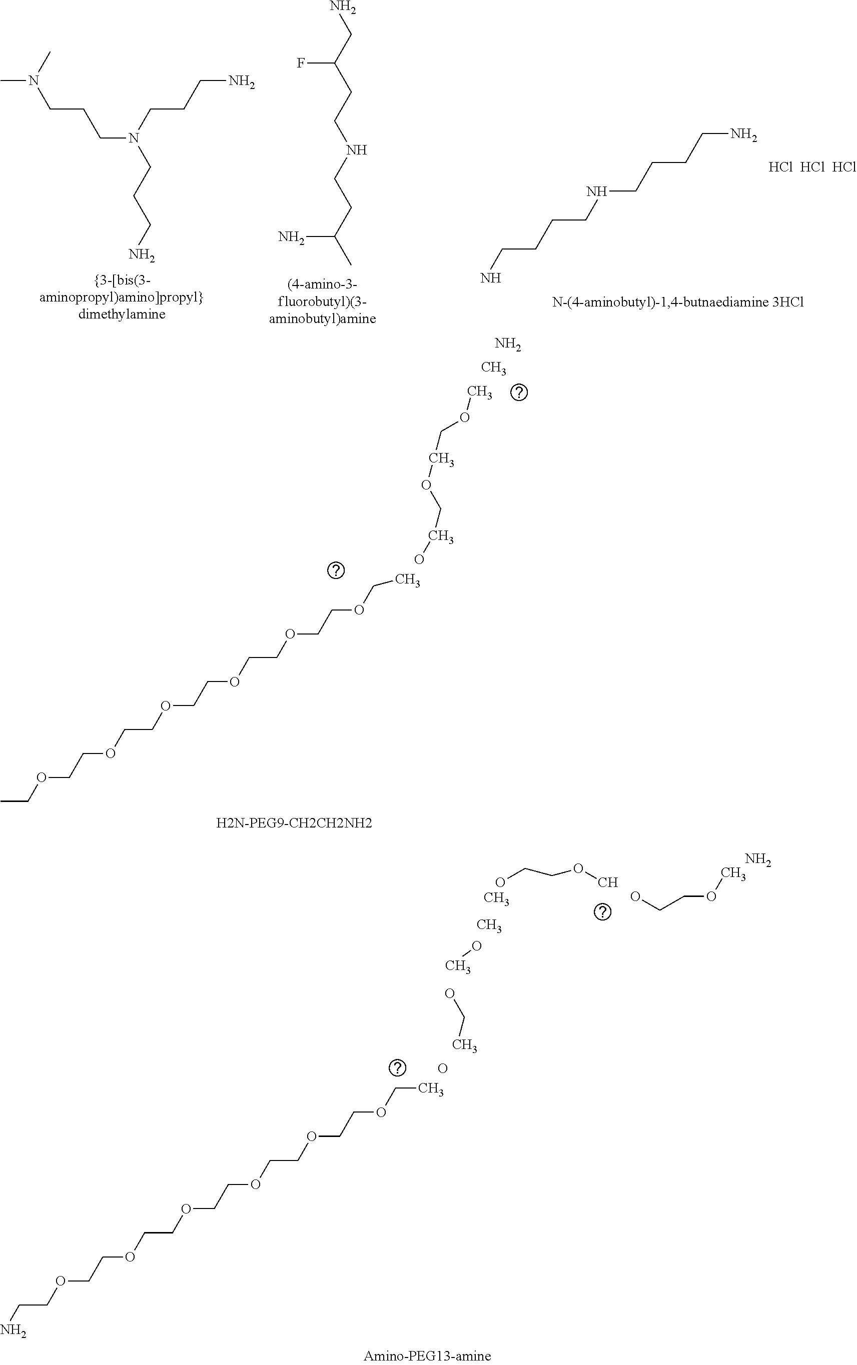

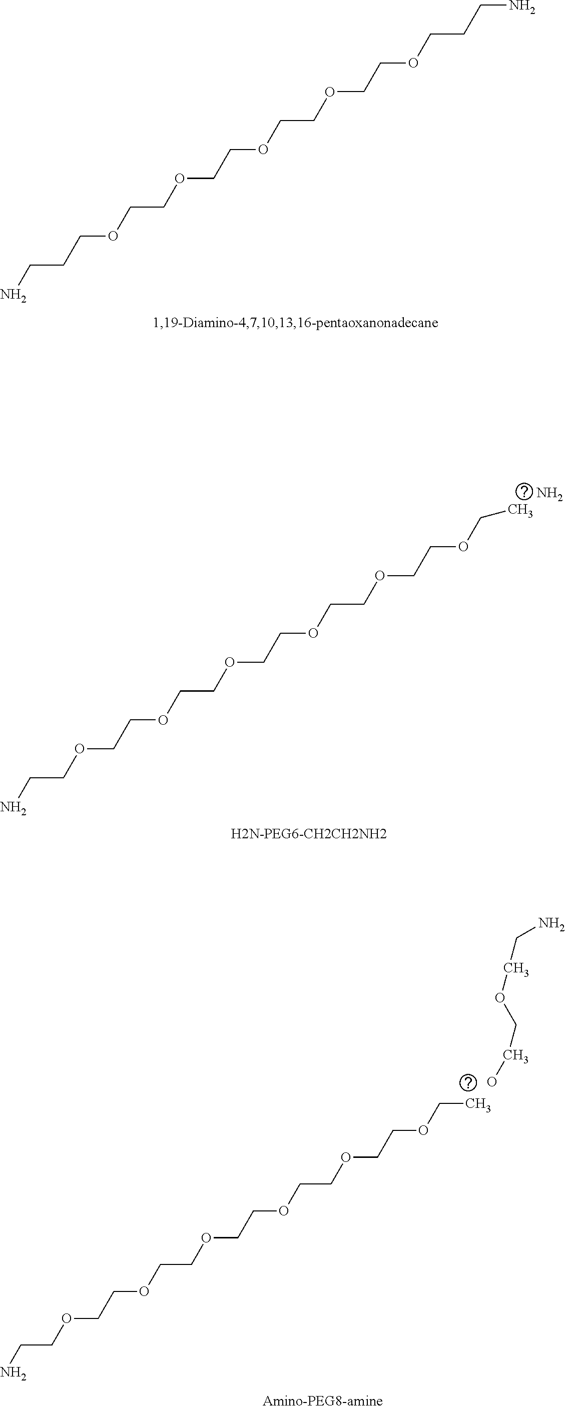

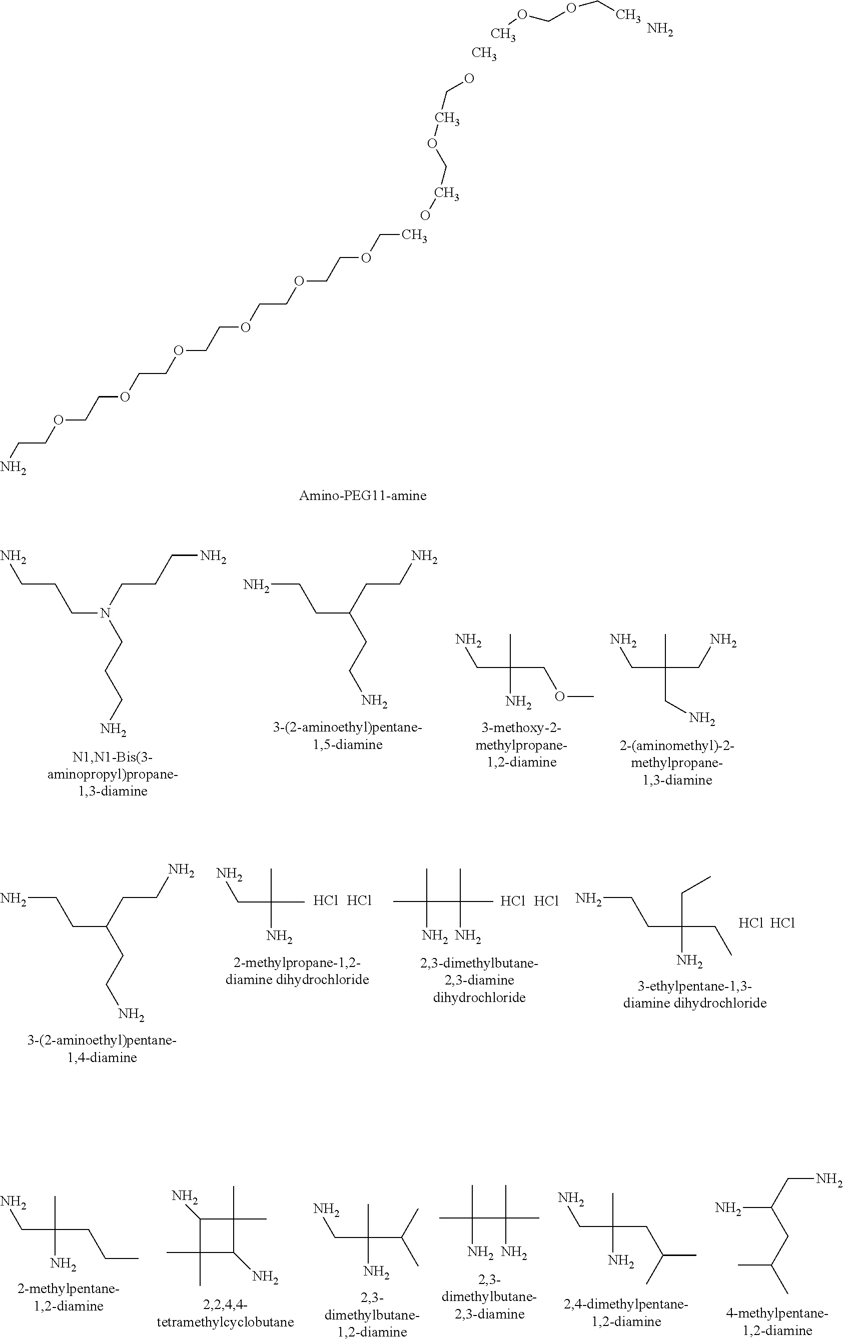

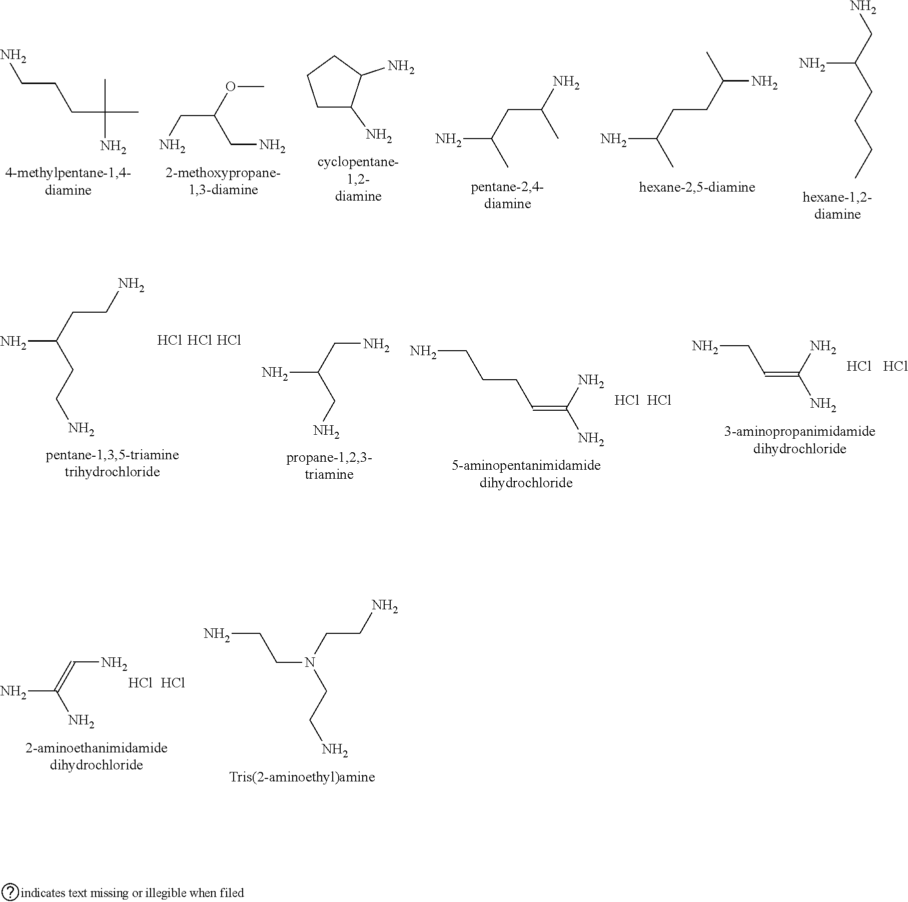

As a nonlimiting illustration of how to create the delivery particle of the invention, when the reaction is stoichiometric, for complete reaction one mole of EDA (two moles of amine) are reacted with two moles of HCTL and one mole of SR610 (two moles of acrylate). In order to have residual acrylate groups for further free-radical polymerization, a 10% molar excess of acrylate, such as SR610 can be utilized (1.1 moles, containing 2.2 moles of acrylate).

### Polymerization Procedure

The initial polymer solution can be prepared as a 20% solution in water. Water is mixed with a magnetic stir bar in a glass beaker. Acrylate is added and dissolved, followed by addition of the HCTL. Finally, an amine, such as EDA is added dropwise to a beaker with continued mixing. The solution is mixed at room temperature for 24 hours to allow complete curing. After 24 hours solution pH is typically around 5.4. Ammonia is added to adjust the pH to alkaline, such as to about 8, and additional mixing is done to allow further curing. The additional cure time at elevated pH enables further reaction of any primary amine reactant species present.

Acidic HCTL lowers the solution pH. After room temperature curing the batch is opaque white, but after pH adjustment and further curing, the solution with these specific materials clarifies slightly and has a pink/tan hue.

The next step of the process is to combine the above ATEC polymer (hydrophilic portion of the surfactant polymer) with an additional (meth)acrylate, such as a multifunctional methacrylate, e.g., (tertiarybutyl aminoethyl methacrylate, TBEAMA), which can form reactive sites for added isocyanate groups

Volume weighted median particle size of delivery particles according to the invention can range from about 1 micron to about 150 microns, or even 5 microns to 150 microns, or even from 10 to 50 microns, preferably 2 to 50 microns, or even 15 to 50 microns.

The isocyanates useful in the invention are to be understood for purposes hereof as isocyanate monomer, isocyanate oligomer, isocyanate prepolymer, or dimer or trimer of an aliphatic or aromatic isocyanate. All such monomers, prepolymers, oligomers, or dimers or trimers of aliphatic or aromatic isocyanates are intended encompassed by the term “isocyanate” as used herein.

The isocyanate is an aliphatic or aromatic monomer, oligomer or prepolymer, usefully of two or more isocyanate functional groups. The isocyanate, for example, can be selected from aromatic toluene diisocyanate and its derivatives used in wall formation for encapsulates, or aliphatic monomer, oligomer or prepolymer, for example, hexamethylene diisocyanate and dimers or trimers thereof, or 3,3,5-trimethyl-5-isocyanatomethyl-1-isocyanato cyclohexane tetramethylene diisocyanate. The polyisocyanate can be selected from 1,3-diisocyanato-2-methylbenzene, hydrogenated MDI, bis(4-isocyanatocyclohexyl) methane, dicyclohexylmethane-4,4′-diisocyanate, and oligomers and prepolymers thereof. This listing is illustrative and not intended to be limiting of the polyisocyanates useful in the invention.

The isocyanates useful in the invention comprise isocyanate monomers, oligomers or prepolymers, or dimers or trimers thereof, having at least two isocyanate groups. Optimal cross-linking can be achieved with isocyanates having at least three functional groups.

Isocyanates, for purposes of the invention, are understood as encompassing any isocyanate monomer, oligomer, prepolymer or polymer having at least two isocyanate groups and comprising an aliphatic or aromatic moiety in the monomer, oligomer or prepolymer. If aromatic, the aromatic moiety can comprise a phenyl, a toluyl, a xylyl, a naphthyl or a diphenyl moiety, more preferably a toluyl or a xylyl moiety. Aromatic polyisocyanates, for purposes hereof, can include diisocyanate derivatives such as biurets and polyisocyanurates. The polyisocyanate, when aromatic, can be, but is not limited to, methylene diphenyl isocyanate, toluene diisocyanate, tetramethylxylidene diisocyanate, polyisocyanurate of toluene diisocyanate (commercially available from Bayer under the tradename Desmodur® RC), trimethylol propane-adduct of toluene diisocyanate (commercially available from Bayer under the tradename Desmodur® L75), or trimethylol propane-adduct of xylylene diisocyanate (commercially available from Mitsui Chemicals under the tradename Takenate® D-110N), naphthalene-1,5-diisocyanate, and phenylene diisocyanate.

Isocyanate, which is aliphatic, is understood as a monomer, oligomer, prepolymer or polymer polyisocyanate which does not comprise any aromatic moiety. There is a preference for aromatic polyisocyanate, however, aliphatic polyisocyanates and blends thereof are useful. Aliphatic polyisocyanates include a trimer of hexamethylene diisocyanate, a trimer of isophorone diisocyanate, a trimethylol propane-adduct of hexamethylene diisocyanate (available from Mitsui Chemicals) or a biuret of hexamethylene diisocyanate (commercially available from Bayer under the tradename Desmodur® N 100).

The capsule shell could also be reinforced using additional co-crosslinkers such as multifunctional amines and/or polyamines such as diethylene triamine (DETA), polyethylene imine, and polyvinyl amine.

### Core

The microcapsules of the present teaching include a benefit agent which comprises one or more ingredients that are intended to be encapsulated. The benefit agent is selected from a number of different materials such as chromogens and dyes, flavorants, perfumes, sweeteners, fragrances, oils, fats, pigments, cleaning oils, pharmaceuticals, pharmaceutical oils, perfume oils, mold inhibitors, antimicrobial agents, fungicides, bactericides, disinfectants, adhesives, phase change materials, scents, fertilizers, nutrients, and herbicides: by way of illustration and without limitation. The benefit agent and oil comprise the core. The core can be a liquid or a solid. With cores that are solid at ambient temperatures, the wall material can usefully enwrap less than the entire core for certain applications where availability of, for example, an agglomerate core is desired on application. Such uses can include scent release, cleaning compositions, emollients, cosmetic delivery, and the like. Where the microcapsule core is phase change material, uses can include such encapsulated materials in mattresses, pillows, bedding, textiles, sporting equipment, medical devices, building products, construction products, HVAC, renewable energy, clothing, athletic surfaces, electronics, automotive, aviation, shoes, beauty care, laundry, and solar energy.

Useful benefit agents include perfume raw materials, such as alcohols, ketones, aldehydes, esters, ethers, nitriles, alkenes, fragrances, fragrance solubilizers, essential oils, phase change materials, lubricants, colorants, cooling agents, preservatives, antimicrobial or antifungal actives, herbicides, antiviral actives, antiseptic actives, antioxidants, biological actives, deodorants, emollients, humectants, exfoliants, ultraviolet absorbing agents, self-healing compositions, corrosion inhibitors, sunscreens, silicone oils, waxes, hydrocarbons, higher fatty acids, essential oils, lipids, skin coolants, vitamins, sunscreens, antioxidants, glycerine, catalysts, bleach particles, silicon dioxide particles, malodor reducing agents, dyes, brighteners, antibacterial actives, antiperspirant actives, cationic polymers and mixtures thereof. Phase change materials useful as benefit agents can include, by way of illustration and not limitation, paraffinic hydrocarbons having 13 to 28 carbon atoms, various hydrocarbons such n-octacosane, n-heptacosane, n-hexacosane, n-pentacosane, n-tetracosane, n-tricosane, n-docosane, n-heneicosane, n-eicosane, n-nonadecane, octadecane, n-heptadecane, n-hexadecane, n-pentadecane, n-tetradecane, n-tridecane. Phase change materials can alternatively, optionally in addition include crystalline materials such as 2,2-dimethyl-1,3-propanediol, 2-hydroxymethyl-2-methyl-1,3-propanediol, acids of straight or branched chain hydrocarbons such as eicosanoic acid and esters such as methyl palmitate, fatty alcohols, and mixtures thereof.

Preferably, in the case of fragrances, a perfume oil acts as benefit agent and solvent for the wall forming material, as illustrated in the examples herein.

Optionally the water phase may include an emulsifier. Non-limiting examples of emulsifiers include water-soluble salts of alkyl sulfates, alkyl ether sulfates, alkyl isothionates, alkyl carboxylates, alkyl sulfosuccinates, alkyl succinamates, alkyl sulfate salts such as sodium dodecyl sulfate, alkyl sarcosinates, alkyl derivatives of protein hydrolyzates, acyl aspartates, alkyl or alkyl ether or alkylaryl ether phosphate esters, sodium dodecyl sulphate, phospholipids or lecithin, or soaps, sodium, potassium or ammonium stearate, oleate or palmitate, alkylarylsulfonic acid salts such as sodium dodecylbenzenesulfonate, sodium dialkylsulfosuccinates, dioctyl sulfosuccinate, sodium dilaurylsulfosuccinate, poly(styrene sulfonate) sodium salt, isobutylene-maleic anhydride copolymer, gum arabic, sodium alginate, carboxymethylcellulose, cellulose sulfate and pectin, poly(styrene sulfonate), isobutylene-maleic anhydride copolymer, carrageenan, sodium alginate, pectic acid, tragacanth gum, almond gum and agar; semi-synthetic polymers such as carboxymethyl cellulose, sulfated cellulose, sulfated methylcellulose, carboxymethyl starch, phosphated starch, lignin sulfonic acid; and synthetic polymers such as maleic anhydride copolymers (including hydrolyzates thereof), polyacrylic acid, polymethacrylic acid, acrylic acid butyl acrylate copolymer or crotonic acid homopolymers and copolymers, vinyl benzenesulfonic acid or 2-acrylamido-2-methylpropanesulfonic acid homopolymers and copolymers, and partial amide or partial ester of such polymers and copolymers, carboxy modified polyvinyl alcohol, sulfonic acid-modified polyvinyl alcohol and phosphoric acid-modified polyvinyl alcohol, phosphated or sulfated tristyrylphenol ethoxylates, palmitamidopropyltrimonium chloride (Varisoft PATC™, available from Degussa Evonik, Essen, Germany), distearyl dimonium chloride, cetyltrimethylammonium chloride, quaternary ammonium compounds, fatty amines, aliphatic ammonium halides, alkyldimethylbenzylammonium halides, alkyldimethylethylammonium halides, polyethyleneimine, poly(2-dimethylamino)ethyl methacrylate) methyl chloride quaternary salt, poly(1-vinylpyrrolidone-co-2-dimethylaminoethyl methacrylate), poly(acrylamide-co-diallyldimethylammonium chloride), poly(allylamine), poly[bis(2-chloroethyl) ether-alt-1,3-bis[3-(dimethylamino)propyl]urea] quaternized, and poly(dimethylamine-co-epichlorohydrin-co-ethyl enediamine), condensation products of aliphatic amines with alkylene oxide, quaternary ammonium compounds with a long-chain aliphatic radical, e.g. distearyldiammonium chloride, and fatty amines, alkyldimethylbenzylammonium halides, alkyldimethylethylammonium halides, polyalkylene glycol ether, condensation products of alkyl phenols, aliphatic alcohols, or fatty acids with alkylene oxide, ethoxylated alkyl phenols, ethoxylated aryl phenols, ethoxylated polyaryl phenols, carboxylic esters solubilized with a polyol, polyvinyl alcohol, polyvinyl acetate, or copolymers of polyvinyl alcohol polyvinyl acetate, polyacrylamide, poly(N-isopropylacrylamide), poly(2-hydroxypropyl methacrylate), poly(-ethyl-2-oxazoline), poly(2-isopropenyl-2-oxazoline-co-methyl methacrylate), poly(methyl vinyl ether), and polyvinyl alcohol-co-ethylene), and cocoamidopropyl betaine. Emulsifier, if employed, is typically from about 0.1 to 40% by weight, preferably 0.2 to about 15% by weight, more typically 0.5 to 10% be weight, based on total weight of the formulation

The microcapsules may encapsulate a partitioning modifier in addition to the benefit agent. Non-limiting examples of partitioning modifiers include isopropyl myristate, mono-, di-, and tri-esters of C₄-C₂₄ fatty acids, castor oil, mineral oil, soybean oil, hexadecanoic acid, methyl ester isododecane, isoparaffin oil, polydimethylsiloxane, brominated vegetable oil, and combinations thereof. Microcapsules may also have varying ratios of the partitioning modifier to the benefit agent so as to make different populations of microcapsules that may have different bloom patterns. Such populations may also incorporate different perfume oils so as to make populations of microcapsules that display different bloom patterns and different scent experiences. US 2011-0268802 discloses other non-limiting examples of microcapsules and partitioning modifiers and is hereby incorporated by reference.

Optionally, if desired, the delivery particles can be dewatered such as through decanting, filtration, centrifuging, or other separation technique. Alternatively, the aqueous slurry delivery particles can be spray dried.

In some examples of the process and compositions, the microcapsules may consist of one or more distinct populations. The composition may have at least two different populations of microcapsules that vary in the exact make-up of the perfume oil and in the median particle size and/or partitioning modifier to perfume oil (PM:PO) weight ratio. In some examples, the composition includes more than two distinct populations that vary in the exact make up the perfume oil and in their fracture strengths. In some further examples, the populations of microcapsules can vary with respect to the weight ratio of the partitioning modifier to the perfume oil(s). In some examples, the composition can include a first population of microcapsules having a first ratio that is a weight ratio of from 2:3 to 3:2 of the partitioning modifier to a first perfume oil and a second population of microcapsules having a second ratio that is a weight ratio of less than 2:3 but greater than 0 of the partitioning modifier to a second perfume oil.

In some embodiments, each distinct population of microcapsules is preparable in a distinct slurry. For example, the first population of microcapsules can be contained in a first slurry and the second population of microcapsules contained in a second slurry. It is to be appreciated that the number of distinct slurries for combination is without limit and a choice of the formulator such that 3, 10, or 15 distinct slurries may be combined. The first and second populations of microcapsules may vary in the exact make up the perfume oil and in the median particle size and/or PM:PO weight ratio.

In some embodiments, the composition, can be prepared by combining the first and second slurries with at least one adjunct ingredient and optionally packaged in a container. In some examples, the first and second populations of microcapsules can be prepared in distinct slurries and then spray dried to form a particulate. The distinct slurries may be combined before spray drying, or spray dried individually and then combined together when in particulate powder form. Once in powder form, the first and second populations of microcapsules may be combined with an adjunct ingredient to form the composition useful as a feedstock for manufacture of consumer, industrial, medical, or other goods. In some examples, at least one population of microcapsules is spray dried and combined with a slurry of a second population of microcapsules. In some examples, at least one population of microcapsules is dried, prepared by spray drying, fluid bed drying, tray drying, or other such drying processes that are available.

In some examples, the slurry or dry particulates can include one or more adjunct materials such as processing aids selected from the group consisting of a carrier, an aggregate inhibiting material, a deposition aid, a particle suspending polymer, and mixtures thereof. Non-limiting examples of aggregate inhibiting materials include salts that can have a charge-shielding effect around the particle, such as magnesium chloride, calcium chloride, magnesium bromide, magnesium sulfate, and mixtures thereof. Non-limiting examples of particle suspending polymers include polymers such as xanthan gum, carrageenan gum, guar gum, shellac, alginates, chitosan; cellulosic materials such as carboxymethyl cellulose, hydroxypropyl methyl cellulose, cationically charged cellulosic materials; polyacrylic acid; polyvinyl alcohol; hydrogenated castor oil; ethylene glycol distearate; and mixtures thereof.

In some embodiments, the slurry can include one or more processing aids, selected from the group consisting of water, aggregate inhibiting materials such as divalent salts; particle suspending polymers such as xanthan gum, guar gum, carboxy methyl cellulose.

In other examples of the invention, the slurry can include one or more carriers selected from the group consisting of polar solvents, including but not limited to, water, ethylene glycol, propylene glycol, polyethylene glycol, glycerol; nonpolar solvents, including but not limited to, mineral oil, perfume raw materials, silicone oils, hydrocarbon paraffin oils, and mixtures thereof.

In some examples, said slurry may include a deposition aid that may comprise a polymer selected from the group comprising: polysaccharides, in one aspect, cationically modified starch and/or cationically modified guar; polysiloxanes; poly diallyl dimethyl ammonium halides; copolymers of poly diallyl dimethyl ammonium chloride and polyvinyl pyrrolidone; a composition comprising polyethylene glycol and polyvinyl pyrrolidone; acrylamides; imidazoles; imidazolinium halides; polyvinyl amine; copolymers of poly vinyl amine and N-vinyl formamide; polyvinyl formamide, polyvinyl alcohol; polyvinyl alcohol crosslinked with boric acid; polyacrylic acid; polyglycerol ether silicone cross-polymers; polyacrylic acids, polyacrylates, copolymers of polyvinylamine and polvyinylalcohol oligomers of amines, in one aspect a diethylenetriamine, ethylene diamine, bis(3-aminopropyl)piperazine, N,N-Bis-(3-aminopropyl)methylamine, tris(2-aminoethyl)amine and mixtures thereof; polyethyleneimine, a derivatized polyethyleneimine, in one aspect an ethoxylated polyethyleneimine; a polymeric compound comprising, at least two moieties selected from the moieties consisting of a carboxylic acid moiety, an amine moiety, a hydroxyl moiety, and a nitrile moiety on a backbone of polybutadiene, polyisoprene, polybutadiene/styrene, polybutadiene/acrylonitrile, carboxyl-terminated polybutadiene/acrylonitrile or combinations thereof; pre-formed coacervates of anionic surfactants combined with cationic polymers; polyamines and mixtures thereof.

In some additional examples to illustrate the invention, at least one population of microcapsules can be contained in an agglomerate and then combined with a distinct population of microcapsules and at least one adjunct material. Said agglomerate may comprise materials selected from the group consisting of silicas, citric acid, sodium carbonate, sodium sulfate, sodium chloride, and binders such as sodium silicates, modified celluloses, polyethylene glycols, polyacrylates, polyacrylic acids, zeolites, and mixtures thereof.

Suitable equipment for use in the processes disclosed herein may include continuous stirred tank reactors, homogenizers, turbine agitators, recirculating pumps, paddle mixers, plough shear mixers, ribbon blenders, vertical axis granulators and drum mixers, both in batch and, where available, in continuous process configurations, spray dryers, and extruders. Such equipment can be obtained from Lodige GmbH (Paderborn, Germany), Littleford Day, Inc. (Florence, Ky., U.S.A.), Forberg AS (Larvik, Norway), Glatt Ingenieurtechnik GmbH (Weimar, Germany), Niro (Soeborg, Denmark), Hosokawa Bepex Corp. (Minneapolis, Minn., U.S.A.), Arde Barinco (New Jersey, U.S.A.).

### TEST METHODS

### Procedure for Determination of % Degradation

% degradation is determined by the “OECD Guideline for Testing of Chemicals” 301B CO₂ Evolution (Modified Sturm Test), adopted 17 Jul. 1992. For ease of reference, this test method is referred to herein as test method OECD 301B

### Procedure for Determination of Free Oil

This method measures the amount of oil in the water phase and uses as an internal standard solution 1 mg/ml dibutyl phthalate (DBP)/hexane.

Weigh a little more than 250 mgs of DBP into a small beaker and transfer to a 250 ml volumetric rinsing the beaker thoroughly. Fill with hexane to 250 ml.

Sample Prep: Weigh approximately 1.5-2 grams (40 drops) of the capsule slurry into a 20 ml scintillation vial and add 10 ml's of the ISTD solution, cap tightly. Shaking vigorously several times over 30 minutes, pipette solution into an autosampler vial and analyze by GC.

Additional details. Instrumentation: HP5890 GC connected to HP Chem Station Software; Column: 5 m×0.32 mm id with 1 μm DB-1 liquid phase; Temperature 50° C.; for 1 minute then heat to 320° C.; @ 15 deg/min; Injector: 275° C.; Detector: 325° C.; 2 ul injection.

Calculation: Add total peak area minus the area for the DBP for both the sample and calibration.)
i) Calculate mg of free core oil:

$$\frac{Total area from sample}{Total area from calibration} \times mg of oil in calibration solution = mg of free oil$$

ii) Calculate % free core oil

$$\frac{mg of free core oil}{Sample wt . ( mg )} \times 100 = \% free core oil in wet slurry$$

### Procedure for Determination of Benefit Agent Leakage

Obtain 2, one-gram samples of benefit agent particle composition. Add 1 gram (Sample 1) of particle composition to 99 grams of product matrix in which the particle will be employed. Age the particle containing product matrix (Sample 1) for 2 weeks at 35° C. in a sealed glass jar. The other one-gram sample (Sample 2) is similarly aged.

After 2 weeks, use filtration to recover the particle composition's particles from the product matrix (Sample 1) and from the particle composition (Sample 2). Treat each particle sample with a solvent that will extract all the benefit agent from each samples' particles. Inject the benefit agent containing solvent from each sample into a Gas Chromatograph and integrate the peak areas to determine the total quantity of benefit agent extracted from each sample.

Determine the percentage of benefit agent leakage by calculating the difference in the values obtained for the total quantity of benefit agent extracted from Sample 2 minus Sample 1, expressed as a percentage of the total quantity of benefit agent extracted from Sample 2, as represented in the equation below:

$$Percentage of Benefit Agent Leakage = ( \frac{Sample 2 - Sample 1}{Sample 2} ) \times 100$$

Delivery particles can be prepared that exhibit positive zeta potentials. Such capsules have improved deposition efficiency, such as on fabrics.

### Sample Preparation for Biodegradability Measurements

The water soluble or water dispersible material is purified via crystallization till a purity of above 95% is achieved and dried before biodegradability measurement.

The oily medium comprising the benefit agent needs to be extracted from the delivery particle slurry in order to only analyze the polymer wall. Therefore, the delivery particle slurry is freeze dried to obtain a powder. Then, it is further washed with organic solvents via Soxhlet extraction method to extract the oily medium comprising the benefit agent till weight percentage of oily medium is below 5% based on total delivery particle polymer wall. Finally, the polymer wall is dried and analyzed.

Weight ratio of delivery particle to solvent is 1:3. Residual oily medium is determined by thermogravimetric analysis (60 minutes isotherm at 100° C. and another 60 minutes isotherm at 250° C.). The weight loss determined needs to be below 5%.

### OECD 301 B—Biodegradability Method

Accumulative CO₂ release is measured over 60 days following the guidelines of the Organisation for Economic Cooperation and Development (OECD)—OECD (1992), Test No. 301: Ready Biodegradability, OECD Guidelines for the Testing of Chemicals, Section 3, OECD Publishing, Paris, https://doi.org/10.1787/9789264070349-en.

### Leakage

The amount of benefit agent leakage from the benefit agent containing delivery particles is determined according to the following method:
i) Obtain two 1 g samples of the raw material slurry of benefit agent containing delivery particles.
ii) Add 1 g of the raw material slurry of benefit agent containing delivery particles to 99 g of the consumer product matrix in which the particles will be employed and label the mixture as Sample 1. Immediately use the second 1 g sample of raw material particle slurry in Step d below, in its neat form without contacting consumer product matrix, and label it as Sample 2.
iii) Age the delivery particle-containing product matrix (Sample 1) for 1 week at 35° C. in a sealed glass jar.
iv) Using filtration, recover the particles from both samples. The particles in Sample 1 (in consumer product matrix) are recovered after the aging step. The particles in Sample 2 (neat raw material slurry) are recovered at the same time that the aging step began for sample 1.
v) Treat the recovered particles with a solvent to extract the benefit agent materials from the particles.
vi) Analyze the solvent containing the extracted benefit agent from each sample, via chromatography.
vii) Integrate the resultant benefit agent peak areas under the curve and sum these areas to determine the total quantity of benefit agent extracted from each sample.
viii) Determine the percentage of benefit agent leakage by calculating the difference in the values obtained for the total quantity of benefit agent extracted from Sample 2 (S2) minus Sample 1 (S1), expressed as a percentage of the total quantity of benefit agent extracted from Sample 2 (s2), as represented in the equation below:

$$\% Leakage = ( \frac{S 2 - S 1}{S 2} ) \times 10$$

### Volume Weighted Mean Particle Size

Particle size is measured using static light scattering devices, such as an Accusizer 780A, made by Particle Sizing Systems, Santa Barbara Calif. The instrument is calibrated from 0 to 300μ using Duke particle size standards. Samples for particle size evaluation are prepared by diluting about 1 g emulsion, if the volume weighted mean particle size of the emulsion is to be determined, or 1 g of benefit agent containing delivery particles slurry, if the finished particles volume weighted mean particle size is to be determined, in about 5 g of de-ionized water and further diluting about 1 g of this solution in about 25 g of water.

About 1 g of the most dilute sample is added to the Accusizer and the testing initiated, using the autodilution feature. The Accusizer should be reading in excess of 9200 counts/second. If the counts are less than 9200 additional sample should be added. The Accusizer will dilute the test sample until 9200 counts/second and initiate the evaluation. After 2 minutes of testing, the Accusizer will display the results, including volume-weighted mean size.

The broadness index can be calculated by determining the particle size at which 95% of the cumulative particle volume is exceeded (95% size), the particle size at which 5% of the cumulative particle volume is exceeded (5% size), and the median particle size (50% size—50% of the particle volume both above and below this size). Broadness Index=((95% size)−(5% size)/50% size).

All percentages and ratios are calculated by weight unless otherwise indicated. All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

In the following examples, the abbreviations correspond to the materials listed in Table 1.

TABLE 1

| Trade | | |
|---|---|---|
| Name/Identifier | | |
| Mondur MR | Company/City | Material |
| Desmodur N3200 | Covestro LLC | Aliphatic polyisocyanate |
| | Baytown, TX | |
| Captex 355/RA | Abitec Corporation, | Medium-chain triglyceride based on caprylic |
| | Columbus, OH | and capric acids |
| SR344 | Sartomer | Polyethylene glycol 400 diacrylate |
| | King of Prussia, PA | |
| AHCTL | | DL-N-acetylhomocysteine thiolactone |
| | | (AHCTL) |
| | | Ethylene diamine |
| Wako V-50 | Wako Specialty | 2,2′-azobis(2-methylpropionamidine) |
| | Chemicals | dihydrochloride |
| | Richmond, VA | |
| SR610 | Sartomer | polyethylene glycol (600) diacrylate |
| | King of Prussia, PA | |
| Vazo 50 | Wako Specialty | 2,2′-azobis(2- |
| | Chemicals | methylpropionamidine)dihydrochloride |
| | North Chsterfield, | |
| | VA | |
| Vazo 88 | Chempoint | 1,1′-azobis(cyanocyclohexane) |
| | Bellevue, WA | |
| HMDA | Dytek, Wilmington, | hexamethylene diamine |
| | DE | |
| SR415 | Sartomer | ethoxylated trimethylolpropane triacrylate |
| | King of Prussia, PA | |
| HCTL | | D,L-homosysteine thiolactone HCl |

### EXAMPLES

### Example 1: In-Situ ATEC

TABLE 2

| Material | Quantity (g) |
|---|---|
| DL-N-Acetylhomocysteine Thiolactone (AHCTL) | 8.92 |
| Dytek HMDA (70% HMDA) | 4.65 |
| SR415 | 21.99 |
| Water | 188.19 |
| Research Accord | 43.53 |
| Captex 355 | 43.53 |

1. Water is placed into jacketed steel reactor and mixed with a flat 4-tip mill blade at 750 rpm and held at 10 C.
2. AHCTL is sprinkled into the water with continued mixing
3. HMDA is added dropwise with continued mixing. The pH before amine addition is 4.0 (with mixing stopped). Max pH is about 10.83.
4. The water phase solution is allowed to mix for 1 hour. Water phase pH is about 9.39 after 1 hour of reaction.
5. Increase the speed of the milling blade to 2000 rpm; add the SR415 slowly with a plastic pipette
6. Increase speed of milling blade to 2500 rpm, Research Accord/Captex core is added dropwise using a plastic pipette.
7. Milling speed is increased to 3500 rpm; temperature is held at 10° C. .
8. Batch is mixed for about 4 hours at 10° C.

The resultant microcapsules are 0.67μ in size (volume-weighted median), exhibit low free core (1.9%) and low LFE leakage (31%).

### Example 2: Interfacial ATEC

TABLE 3

| Material | Quantity (g) |
|---|---|
| DL-N-Acetylhomocysteine Thiolactone (AHCTL) | 8.92 |
| Dytek HMDA (70% HMDA) | 4.65 |
| SR415 | 21.99 |
| Water | 188.19 |
| Research Accord | 43.53 |
| Captex 355 | 43.53 |

1. Water is placed into a jacketed steel reactor and mixed with a flat 4-tip mill blade at 750 rpm and held at 20 C.
2. AHCTL is sprinkled into the water with continued mixing
3. HMDA is added dropwise with continued mixing. The pH before amine addition is 4.94 (with mixing stopped). Max pH is about 10.40.
4. The water phase solution is allowed to mix for 3 hours. Water phase pH is about 8.83 after 3 hours of reaction.
5. The Research Accord, Captex and SR415 are combined in a glass beaker and mixed until the solution is clear.
6. Core solution is added dropwise to the water phase via pipette with mixing at 2000 rpm. This addition takes about 30-35 minutes. A white emulsion is created while adding the oil phase, but no thickening is seen during the addition. Temperature is held at 20° C., mixing speed is increased to 3000 rpm after all of oil phase is added. Emulsion is bright white in color with no thickening and no chunks seen. The batch is covered and allowed to mix at 3000 rpm overnight.
7. Batch is mixed for about 17 hours at room temperature.

The resultant microcapsules have a volume-weighted median particle size of 5.35μ, free core of 2.4% and LFE leakage of 60%.

### Example 3: ATEC/PU Polymer Preparation, Capsule Batch Preparation

For this process, an ATEC polymer emulsifier/cross-linker is created in the first polymer preparation step, followed by the use of the polymer in a microencapsulation process, in which the polymer acts as the only emulsifier in the process and also cross-links the polyurea wall material.

### Polymer Preparation

TABLE 4

| Material | Quantity (g) |
|---|---|
| DL-N-Acetylhomocysteine Thiolactone (AHCTL) | 4.55 |
| D,L-Homocysteine Thiolactone (HCTL) | 4.02 |
| SR610 | 19.31 |
| Water | 160.32 |
| Bis(hexamethylene)triamine (BHMT) | 11.80 |
| (50:50 BHMT/water solution | |

1. The water is added to a 250 g beaker and stirred with a magnetic stir bar.
2. The SR610 is added and stirred to dissolve.
3. Next the HCTL and then the AHCTL is added and mixed. After the HCTL/AHCTL additions, solution pH is about 3.87.
4. The BHMT is added and mixed with a magnetic stir bar. During the BHMT addition, pH maxes out at about 10.35 and then begins to drop. The pH has dropped to about 7.99 after 1 hour.
5. The pH is adjusted back up to 9 using 30% ammonia (0.79 g).
6. After 2 hours pH is back down to about 8.56 and is adjusted back again to 9 with 30% ammonia (0.42 g).
7. The solution is covered and allowed to stir at room temperature for about 24 hours.

Polymer pH is about 8.26 after reaction. Polymer is pH adjusted to 5.84 (aim for 5.84) with 1.85 g of HCL. The solution is slightly viscous, golden yellow in color and becomes slightly foamy when mixed.

### Microcapsule Batch Preparation

TABLE 5

| Material | Quantity (g) |
|---|---|
| Water Phase Polymer (prepared in previous step) | 200 |
| Oil Phase: | |
| Captex 355 | 195.69 |
| Mondur MR | 3.74 |
| Desmodur N3200 | 0.57 |

1. Water phase is added to jacketed reactor 1 at 22° C. with 1000 rpm mixing (4-tip flat mill). The mixer is offset on the side of the reactor, with the blade nearly touching the side.
2. The oil phase (prepared in advance and mixed at room temperature with a magnetic stir bar), is added to the mixing water phase via plastic pipette. Mixing is initially set at 2000 rpm and is increased to 2500 rpm after about half the core is added.
3. Milling was increased to 4000 rpm and was maintained at 22 C throughout.
4. After milling, the emulsion is mixed with a 3″ propeller at 400 rpm.
5. The emulsion is brought up to a pH of 6.00 using Caustic Soda prior to the start of the curing phase
6. The batch is heated from 22 C to 65 C in 30 minutes and held at 65 C for 4 hours.
7. Batch is cooled back to room temperature naturally.
8. After milling the pH of the emulsion is 4.76, the emulsion is brought back up to a pH of 6.03 (aiming for 6.00) using 0.73 g of Caustic Soda (Decom part #902-167).

Final microcapsule size (vol-wt median) is 6.52μ, free core is 0.5% and LFE leakage is 33%.

### Example 4: ATEC/Acrylate/PU

This process utilizes both ATEC and standard free-radical acrylate chemistry to form the polymer emulsifier used in the polyurea microencapsulation process. The ATEC polymer contains residual acrylate functionality which are used as reactive sites for the subsequent acrylate reaction. In addition to acting as an emulsifier, the polymer emulsifier contains amine functionality that is used to cross-link the isocyanate wall material.

### Polymer Preparation—ATEC Polymer

TABLE 6

| Material | Quantity (g) |
|---|---|
| DL-N-Acetylhomocysteine Thiolactone (AHCTL) | 4.22 |
| Ethylene Diamine (EDA) | 0.87 |
| SR344 | 7.14 |
| Water | 183.8 |
| Research Accord | 43.53 |
| Captex 355 | 43.53 |

1. The water is added to a 250 g beaker and stirred with a magnetic stir bar.
2. The SR344 is added and stirred to dissolve.
3. Next the AHCTL is added and mixed. After the AHCTL addition, solution pH is about 3.99. 30% ammonia is added to adjust the pH to about 9 before the EDA addition (0.08 g ammonia solution added).
4. Finally the EDA is added and mixed. EDA is added during mixing with a magnetic stir bar. After the EDA addition, pH is about 10.25. The pH drops back to low 9s rapidly so 2.33 g of 30% ammonia is added to about pH 10.
5. The solution is mixed at room temperature overnight.

The polymer solution is clear, with just a few particles visible. The solution is foamy when agitated vigorously. Polymer pH is 9.50.

### Free-Radical Acrylate Reaction on ATEC Polymer

The A-TEC solution prepared above is placed in a jacketed steel reactor at 40° C., with a nitrogen blanket at 100 cc/min, and mixing at 1000 rpm with a 4-tip flat mill blade. Another water solution is prepared in a beaker with the composition shown in table 7 below.

TABLE 7

| Material | Quantity (g) |
|---|---|
| Wako V-50 | 0.2 |
| TBAEMA | 3.84 |

The A-TEC polymer from the previous step is placed into a jacketed steel reactor, at 40 C, with a nitrogen blanket at 100 cc/min, and with mixing at 750 rpm (4-tip flat mill).
1. The V-50 and TBAEMA are added directly to the reactor, with the TBEMA being added dropwise while mixing is maintained at 750 rpm.
2. The solution is heated from 40° C. to 75° C. in 30 minutes, held at 75° C. for 8 hours and then cooled back to 25° C.

TABLE 8

| Material | Quantity (g) |
|---|---|
| Water Phase Polymer (prepared in previous step) | 200 |
| Oil Phase: | |
| Captex 355 | 200 |
| Mondur MR | 4.0 |

### Microcapsule Preparation

1. Water phase is added to jacketed reactor 1 at 20° C. with 1000 rpm mixing (4-tip flat mill). The mixer is offset on the side of the reactor, with the blade nearly touching the side.
2. The oil phase (prepared in advance and mixed at room temperature with a magnetic stir bar), is added to the mixing water phase via plastic pipette. Mixing is initially at 2000 rpm and is increased to 2500 rpm after about half the core is added.
3. Milling is increased to 4000 rpm and the emulsion is maintained at 20° C. throughout.
4. After milling, the emulsion is mixed with a 3″ propeller at 425 rpm.
5. The batch is heated from 20° C. to 50° C. in 30 minutes and held at 50° C. for 8 hours.
6. Batch is cooled back to room temperature naturally.

The finished microcapsules have a volume-weighted median particle size of 15.8μ and have a free core of less than 1%.

### Example 5: ATEC/Acrylate

Polymer preparation is done identically to Example 3 (above) but SR610 is 19.31 g (a 10% acrylate excess). The resultant polymer emulsifier contains residual acrylate functionality which is able to further react with acrylate microcapsule wall originating from the oil phase.

200 g of the polymer surfactant is placed in a beaker and Wako V50 initiator (0.3 g) is added and dissolved in the water phase with mixing (magnetic stir bar). The water phase is heated to 50 C about 30 minutes before use.

A 50/50 mixture of limonene and Captex 355 (100 g) is added to a steel jacketed reactor at 35C. A nitrogen blanket is applied at 100 cc/min. Mixing is done at 300 rpm with a flat 4-tip mill blade. Vazo 67 (0.3 g) and Vazo 88 (0.3 g) are added and dissolved. The reactor is heated from 35 to 70 C in 45 minutes, held at 70 C for 45 minutes, and cooled to 50 C in 45 minutes. A second oil phase (15 g of 50/50 limonene/Captex and 10 g of Sartomer SR206) is added and mixing is continued at 50 C for 60 minutes. Mixing is stopped, the pre-heated water phase is added, and milling is started (3000 rpm for 60 minutes, while temperature is maintained at 50 C). Size may be adjusted by varying milling speed and time. After milling is completed, the mill blade is replaced by a 3″ propeller mixer running at 300 rpm. The batch is heated from 50 to 70 C, held at 70 C for 4 hours, heated from 70 C to 95 C, and held at 95 C for 6 hours before naturally cooling back to room temperature. The produced microcapsules are useful for fragrance, agriculture, and phase change applications.

Percent degradation is measured according to the OECD Guidelines for the Testing of Chemicals, test method OECD 301B. A copy is available in www.oecd-ilibrary.org.

Capsules according to the invention can have core to wall ratios even as high as 95% core to 1% wall by weight. In applications where enhanced degradability is desired, higher core to wall ratios can be used such as 99% core to 1% wall, or even 99.5% to 0.5% by weight or higher. With appropriate selection of core to wall ratios, the shell of the composition according to the invention can be selected to achieve a % degradation of at least 40% degradation after 14 days, of at least 50% degradation after at least 20 days, and of at least 60% degradation after at least 28 days when tested according to test method OECD 301B.

Uses of singular “a,” “an,” are intended to cover both the singular and the plural, unless otherwise indicated herein or clearly contradicted by context. The terms “comprising,” “having,” “including,” and “containing” are to be construed as open-ended terms. All references, including publications, patent applications, and patents, cited herein are hereby incorporated by reference. Any description of certain embodiments as “preferred” embodiments, and other recitation of embodiments, features, or ranges as being preferred, or suggestion that such are preferred, is not deemed to be limiting. The invention is deemed to encompass embodiments that are presently deemed to be less preferred and that may be described herein as such. All methods described herein can be performed in any suitable order unless otherwise indicated herein or otherwise clearly contradicted by context. The use of any and all examples, or exemplary language (e.g., “such as”) provided herein, is intended to illuminate the invention, and does not pose a limitation on the scope of the invention. Any statement herein as to the nature or benefits of the invention or of the preferred embodiments is not intended to be limiting. This invention includes all modifications and equivalents of the subject matter recited herein as permitted by applicable law. Moreover, any combination of the above-described elements in all possible variations thereof is encompassed by the invention unless otherwise indicated herein or otherwise clearly contradicted by context. The description herein of any reference or patent, even if identified as “prior,” is not intended to constitute a concession that such reference or patent is available as prior art against the present invention. No unclaimed language should be deemed to limit the invention in scope. Any statements or suggestions herein that certain features constitute a component of the claimed invention are not intended to be limiting unless reflected in the appended claims.

What is claimed is:

1. A delivery particle comprising a core material and a shell encapsulating the core material, wherein the core material comprises a benefit agent, and,
wherein the shell comprises a polymer, the polymer comprising a reaction product of:
an amine-thiol-ene conjugate.

2. The delivery particle according to claim 1 wherein the amine of the amine-thiol-ene conjugate is a mono or bis alkyl diamine, or mono or bis alkyl triamine.

3. The delivery particle according to claim 1 wherein the thiol of the amine-thiol-ene conjugate is a thiolactone.

4. The delivery particle according to claim 3 wherein the thiol of the amine-thiol-ene conjugate is selected from the group consisting of a homocysteine thiolactone and an acetylated homocysteine thiolactone.

5. The delivery particle according to claim 1 wherein the ene of the amine-thiol-ene conjugate is an unsaturated compound selected from the group consisting of a vinyl compound and an acrylate.

6. The delivery particle according to claim 1 wherein the amine-thiol-ene conjugate is in addition a free-radically crosslinked reaction product of the amine-thiol-ene conjugate with one or more of a multifunctional (meth)acrylate and an isocyanate.

7. The delivery particle according to claim 1 wherein the amine-thiol-ene conjugate is in addition a reaction product of the amine-thiol-ene conjugate with a multifunctional (meth)acrylate.

8. The delivery particle according to claim 5 wherein the vinyl compound in addition includes a functional group comprising an electron withdrawing group.

9. The delivery particle according to claim 1 wherein the amine-thiol-ene conjugate in addition comprises a reaction product of the amine-thiol-ene conjugate crosslinked with an isocyanate.

10. The delivery particles according to claim 9 wherein the reaction product of the amine-thiol-ene conjugate with an isocyanate is further free-radically crosslinked with a multifunctional (meth)acrylate.

11. The delivery particle according to claim 1 wherein the amine-thiol-ene conjugate comprises a further crosslinked reaction product with one or more of an isocyanate, a Michael adduct comprising a multifunctional (meth)acrylate, or one or more of an aza-Michael adduct comprising a multifunctional acrylate.

12. The delivery particle according to claim 1 comprising a core material and a shell encapsulating the core material, wherein the core material comprises a benefit agent, and,
wherein the shell comprises a polymer, the polymer comprising a reaction product of:
an amine, a thiolactone, a multifunctional (meth)acrylate and a polyfunctional isocyanate.

13. The delivery particle according to claim 1 wherein the amine-thiol-ene conjugate is the reaction product of an alkyl amine, a 4-, 5-, or 6-member thiolactone, and a vinyl compound having an electron withdrawing group.

14. The delivery particle according to claim 13 wherein the alkyl amine is selected from a 3 to 18 carbon alkyl moiety.

15. The delivery particle according to claim 1 wherein the thiolactone is selected from homocysteine-γ-thiolactone, acetylhomocysteine thiolactone, α-amino-γ-butyrothiolactone, N-acetylhomocysteine thiolactone, a-isocyanato-γ thiolactone, or N-(2-alkylacetyl)homocysteine thiolactone.

16. The delivery particle according to claim 1 wherein the molar ratio of the amine to thiol to ene moieties in the conjugate is from 75/1/1.25 to 1.25/1/0.75 preferably 0.85/1/1.15 to 1.15/1/0.85 more preferably 0.95/1/1.05 to 1.05/1/0.95.

17. The delivery particle according to claim 10 wherein the multifunctional (meth)acrylate is present in a molar excess as compared to the thiolactone and amine, and is selected from an oil soluble (meth)acrylate selected from group consisting of a bi-functional (meth)acrylate, a tri-functional (meth)acrylate, a tetra-functional (meth)acrylate, a penta-functional (meth)acrylate, a hexa-functional (meth)acrylate, a hepta-functional (meth)acrylate, an octa-functional (meth)acrylate and mixtures thereof.

18. The delivery particle according to claim 1 wherein the multifunctional (meth)acrylate is selected from a water soluble or dispersible (meth)acrylate selected from 2-carboxyethyl acrylate, 2-carboxyethyl acrylate oligomers, 2-carboxypropyl acrylate, 4-acryloyloxyphenylacetic acid, carboxyoctyl acrylate, tripropylene glycol diacrylate, ethoxylated bisphenol diacrylate, dipropylene glycol diacrylate, alkoxylated hexanediol diacrylate, alkoxylated cyclohexane dimethanol diacrylate, propoxylated neopentyl glycol diacrylate, trimethylolpropane triacrylate, pentaerythritol triacrylate, ethoxylated trimethylolpropane triacrylate, propoxylated trimethylolpropane triacrylate, propoxylated glyceryl triacrylate, ditrimethylolpropane tetraacrylate, dipentaerythritol pentaacrylate, ethoxylated pentaerythritol tetraacrylate, glycerol tri(meth)acrylate, ethylene glycol diacrylate, di-, tri-, tetra-, or pentaethylene glycol diacrylate, dipropylene glycol diacrylate, polyethylene glycol diacrylate, 2-ethylhexyl acrylate, 2-hydroxyethyl (meth)acrylate, cyanoethyl acrylate, 2-hydroxypropyl acrylate, lauryl acrylate, cyclohexyl acrylate, tetrahydrofurfuryl acrylate, chlorobenzyl acrylate, amino alkylacrylate, ethylaminoethyl (meth)acrylate, aminoethyl (meth)acrylate, tertiarybutyl aminoethyl (meth)acrylate, diethylamino (meth)acrylate, diethylaminoethyl (meth)acrylate, dimethylaminoethyl (meth)acrylate independently or a combination of the foregoing.

19. The delivery particle according to claim 1 wherein the shell in addition comprises an isocyanate compound, the isocyanate compound being aliphatic or aromatic and selected from one or more of an isocyanate monomer, oligomer or prepolymer, dimer or trimer, having at least two isocyanate groups.

20. The delivery particle of claim 1, wherein the benefit agent is a fragrance, preferably a fragrance comprising perfume raw materials characterized by a logP of from about 2.5 to about 4.5.

21. The delivery particle of claim 1, wherein the core comprises in addition a partitioning modifier selected from the group consisting of isopropyl myristate, vegetable oil, modified vegetable oil, mono-, di-, and tri-esters of C4-C24 fatty acids, dodecanophenone, lauryl laurate, methyl behenate, methyl laurate, methyl palmitate, methyl stearate, and mixtures thereof, preferably isopropyl myristate.

22. The delivery particle of claim 1, wherein the wall has a biodegradability above 30% CO2 in 60 days following OECD 301B test, preferably above 40% CO2, more preferably above 50% CO2, even more preferably above 60% CO2 (maximum 95%).

23. The delivery particle according to claim 1, wherein the wall of the delivery particles further comprises a coating material, preferably wherein the coating material is selected from the group consisting of poly(meth)acrylate, poly(ethylene-maleic anhydride), polyamine, wax, polyvinylpyrrolidone, polyvinylpyrrolidone co-polymers, polyvinylpyrrolidone-ethyl acrylate, polyvinylpyrrolidone- vinyl acrylate, polyvinylpyrrolidone methacrylate, polyvinylpyrrolidone/vinyl acetate, polyvinyl acetal, polyvinyl butyral, polysiloxane, poly(propylene maleic anhydride), maleic anhydride derivatives, co-polymers of maleic anhydride derivatives, polyvinyl alcohol, styrene-butadiene latex, gelatine, gum arabic, carboxymethyl cellulose, carboxymethyl hydroxyethyl cellulose, hydroxyethyl cellulose, other modified celluloses, sodium alginate, chitosan, chitin, casein, pectin, modified starch, polyvinyl acetal, polyvinyl butyral, polyvinyl methyl ether/maleic anhydride, polyvinyl pyrrolidone and its co polymers, poly(vinyl pyrrolidone/methacrylamidopropyl trimethyl ammonium chloride), polyvinylpyrrolidone/vinyl acetate, polyvinyl pyrrolidone/dimethylaminoethyl methacrylate, polyvinyl amines, polyvinyl formamides, polyallyl amines, copolymers of polyvinyl amines, and mixtures thereof.

24. The delivery particle according to claim 1, wherein the delivery particle has a leakage of below about 50%, or at most about 50% as determined by the Leakage Test described in the TEST METHODS Section.

25. The delivery particle according to claim 1 wherein the delivery particle has a volume weighted median particle size of from 5 microns to 150 microns, or even from 10 to 50 microns, or even from 15 to 50 microns.

26. An article of manufacture incorporating the delivery particles according to claim 1.

27. The article of manufacture according to claim 26 wherein the article is selected from the group consisting of an agricultural formulation, a slurry encapsulating an agricultural active, a population of dry microcapsules encapsulating an agricultural active, an agricultural formulation encapsulating an insecticide, and an agricultural formulation for delivering a preemergent herbicide.

28. The article of manufacture according to claim 26 wherein the agricultural active is selected from the group consisting of an agricultural herbicide, an agricultural pheromone, an agricultural pesticide, an agricultural nutrient, an insect control agent, and a plant stimulant.
